(19) United States
(12) Patent Application Publication
(10) Pub. No.: US 2001/0043887 A1
(43) Pub. Date: Nov. 22, 2001

(54) High mass-flow rate air purifier

(76) Inventors: Guy G. Morneault, Kelowna (CA); Stephen G. Morneault, Kelowna (CA)

Correspondence Address: ANTONY C. EDWARDS, BISHOP & COMPANY, SUITE 206, 347 LEON AVENUE, KELOWNA, BC, V1Y 8C7, CA

(21) Appl. No.: 09/855,546

(22) Filed: May 16, 2001

Related U.S. Application Data
(60) Provisional application No. 60/205,079, filed on May 18, 2000.

(51) Int. Cl. A61L 9/20

(52) U.S. Cl. 422/121; 96/224

(57) ABSTRACT

An air purifier includes a high mass-flow rate air-mover and a low mass-flow rate ultra-violet radiation decontamination device. The decontamination device is mounted in spaced relation to the air-mover so as to space the inlet of the air-mover from the inlet of the decontamination device. In particular, the inlet of the decontamination device may be disposed oppositely to the inlet of the air-mover, relative to the outlet of the decontamination device, along a low mass-flow rate flow path of a low mass-flow rate air flow. The low mass-flow rate air flow passes ambient air into the inlet of the decontamination device, through the decontamination device so as to pass for an operative dwell time into operative proximity to at least one UV emitter mounted in the decontamination device and so as to exit from the outlet of the air-mover to then be drawn into the inlet of the air-mover. The air-mover is spaced from the decontamination device so as to draw into the inlet of the air-mover a second air-flow of the ambient air. The second air flow flows along a second flow path which does not flow within the operative proximity to any UV emitter in the decontamination device.

### CROSS REFERENCE TO RELATED APPLICATION

This application claims priority from U.S. Provisional Patent Application No. 60/205,079 filed May 18, 2000 entitled High Air-Mass Flow Rate Air Purifier.

### FIELD OF THE INVENTION

This invention relates to an apparatus that will decontaminate and purify large volumes of air such as may be required during the restoration of a building after fire or flood damage.

### BACKGROUND OF THE INVENTION

The generally accepted method of building decontamination for relatively minor occurrences is to simply increase through ventilation of air from outside. Where contamination is much more extensive such as that resulting from a small fire or from water damage, building decontamination is somewhat more efficient when large volume air blowers, such as the Hurricane™ available from Dry-Ease™ of Mount Vernon, Wash., are used to simply increase the circulation of air from the building to the outside. However, the problem associated with eliminating noxious odours and chemical contaminants resulting from burned and singed material and the growth of mould and other organisms within soaked building materials and furniture is not addressed. Remedial action for these factors generally results in a significant cost and time delay prior to reoccupancy.

In the prior art it is known that an improvement of the air quality of air moving within air ducts within residences and commercial buildings can be achieved by the use of ultraviolet radiation to treat the moving column of air. One such device is the subject of U.S. Pat. No. 5,833,740 to Brais, which issued Nov. 10, 1998, and sold commercially by Sanuvox Technologies Inc. of Montreal, Quebec. The drawback of such devices is that they are most efficient at treating only low mass-flow rates. For the Sanuvox S1000™ model this is taught to be 99.6% bacteria kill per pass at a 209 ms UV exposure time (150 cfm), dropping to 98.8% bacteria kill per pass at a 43 ms UV exposure time (1000 cfm). Large volume air blowers however have typically 2000-2500 cfm capacities.

Therefore a need exists, and it is an object of the present invention to provide, a means of adapting a low mass-flow rate UV purifier such as supplied by Sanuvox for use with a large volume air moving blower, for example such as is in common usage during fire or flood restoration, to eliminate from the air within a building particulates from combustion, carbon monoxide, chemical contaminants and micro-organisms such as molds and fungi.

The velocity of air movement past an ultraviolet source is critical since the reduction of contaminants is proportional to the intensity of the ultraviolet source and the length of time the contaminants are exposed to the ultraviolet source. Thus for example, coupling a large volume air-moving blower to the device of Brais, while maintaining a low air flow rate past the ultraviolet source of the device has not, to the applicant's knowledge, been successfully done in the past.

Generally, it is one object of this invention, without intending to be limiting, to provide a means where a commercially available air purifier utilising a source of ultraviolet radiation may be adapted for use with a commercially available air blower of a type which is normally used for venting and drying the interior of a building during fire or flood restoration.

Without intending to be limiting, a further object of this invention is to provide a portable device whereby a large volume of air within a structure may be continuously recycled past a source of ultraviolet radiation so that the air within the structure can be expeditiously purified for a specific range of chemical and biological contaminants.

### SUMMARY

The air purifier of the present invention includes a high mass-flow rate air-mover and a low mass-flow rate ultra-violet radiation decontamination device. The decontamination device is mounted in spaced relation to the air-mover so as to space the inlet of the air-mover from the inlet of the decontamination device. In particular, the inlet of the decontamination device may be disposed oppositely to the inlet of the air-mover, relative to the outlet of the decontamination device, along a low mass-flow rate flow path of a low mass-flow rate air flow. The low mass-flow rate air flow passes ambient air into the inlet of the decontamination device, through the decontamination device so as to pass for an operative dwell time into operative proximity to as least one UV emitter mounted in the decontamination device and so as to exit from the outlet of the air-mover to then be drawn into the inlet of the air-mover. The air-mover is spaced from the decontamination device so as to draw into the inlet of the air-mover a second air flow of ambient air. The second air flow flows along a second flow path which does not flow within the operative proximity to any UV emitter in the decontamination device.

The low mass-flow rate air flow and the second air flow cumulatively form a high mass-flow rate flow being urged by the air-mover through the air-mover so as to expel the high mass-flow rate air flow into the ambient air.

In one embodiment, the air-mover and the decontamination device are rigidly mounted vertically spaced from one another within a housing. The decontamination device may be mounted above or below the air-mover. The air-mover may be a blower and the UV emitter may be mounted within a duct.

The housing may have an outlet aperture in a first surface of the housing and at least one inlet aperture in a second surface of the housing, the housing otherwise being substantially sealed to air flow. The outlet aperture communicates with the outlet of the air-mover. The inlet aperture or apertures are adjacent the inlet of the decontamination device and communicate ambient air flow into both the second flow path and the low mass-flow rate flow path.

In a preferred embodiment, the outlet aperture does not face in the same direction as the inlet apertures. Thus air flow from the outlet aperture exhausting into the ambient air may recirculate within the air space of an enclosure in which the air purifier is placed before being re-drawn as ambient air flow into the inlet apertures.

The decontamination device duct is aligned so that the low mass-flow rate air flow through the duct is orthogonal to a plane substantially containing the inlet of the air-mover. The duct may be elongate and aligned orthogonally to an exhaust direction of the blower.

In one embodiment the inlet apertures are generally perpendicular to the outlet aperture and may be formed in side walls of the housing. The inlet apertures may be an array of apertures vertically spaced over one sidewall of the housing.

In embodiments of the invention, the second air flow and the low mass-flow rate air flow may be generally parallel at positions along the air flows when the low mass-flow rate air flow is in operative proximity to a UV emitter in the decontamination device.

The second air flow and the low mass-flow rate air flow may be ducted in a common duct containing at least one UV emitter. The decontamination device and the air-mover may be co-axial along the substantial air flow directions of the second air flow, the low mass-flow rate air flow, and an exhaust air flow direction from the outlets of the air-mover and the decontamination device.

### BRIEF DESCRIPTION OF THE DRAWINGS

FIG. 1 is an exploded isometric view of one embodiment of the air purifier of the present invention.

FIG. 2 is an exploded side elevation view of the air purifier of FIG. 1.

FIG. 3 is a side elevation view of the assembled air purifier of FIGS. 1 and 2.

FIG. 4 is an enlarged partial sectional view of the outlet end of the air purifier of the present invention in an alternative embodiment.

FIG. 5 is an end elevation view of the outlet end of FIG. 4.

FIG. 6 is an enlarged partial sectional view of the outlet end of a further alternative embodiment of the present invention.

FIG. 7 is an enlarged partial sectional view of an outlet end of the present invention in a further alternative embodiment of the invention.

FIG. 8 is an end elevation view of the outlet end of FIG. 7.

FIG. 9 is, in perspective exploded view, an alternative embodiment of the air purifier of the present invention.

FIG. 10 is a sectional view along line 10-10 in FIG. 9.

FIG. 11 is a sectional view along line 11-11 in FIG. 10.

FIG. 12 is the view of FIG. 10 showing an illustrative air flow pattern through the housing.

FIG. 13 is a partially cut-away enlarged view of the air purifier of FIG. 11 in a further alternative embodiment.

FIG. 14 is, in partially exploded perspective view, a further alternative embodiment of the air purifier of the present invention.

FIG. 15 is a sectional view along line 15-15 in FIG. 14.

### DETAILED DESCRIPTION OF EMBODIMENTS OF THE INVENTION

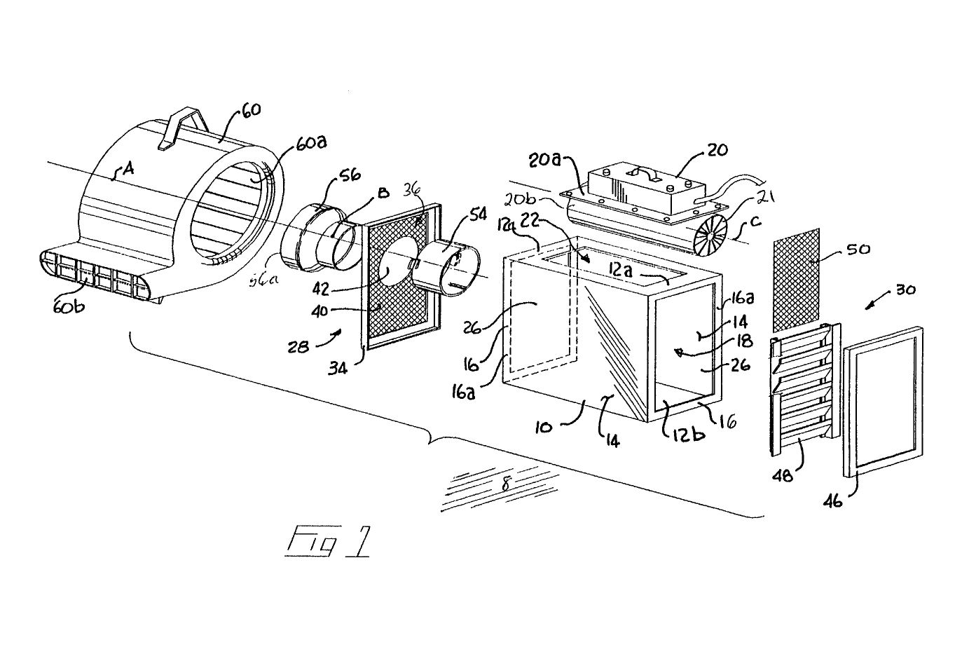

As illustrated in FIGS. 1-3, in a first embodiment of the air purifier of the present invention, a generally rectangular housing 10, advantageously formed from sheet metal or like material that will not degrade under long exposure to ultraviolet radiation, has upper and lower walls 12a and 12b respectively, contiguous side walls 14 and end walls 16. The walls of housing 10 enclose a cavity 18 within which an ultraviolet radiation emitting device, hereinafter UV device 20, such as the device which is the subject of U.S. Pat. No. 5,833,740 may be housed. UV device 20 may have a flow rate capacity of approximately 144 cfm.

Upper wall 12a may be formed as an inwardly projecting perimeter lip surrounding an aperture 22 into which UV device 20 may be mounted. In one form, UV device 20 has an outwardly projecting flange 20a which permits secure mounting of UV device 20 to upper wall 12a. End walls 16 are each formed as inwardly projecting perimeter lips 16a so as to define opposed apertures 26 in each of end walls 16. Apertures 26 are closed by front and rear cover members 28 and 30 respectively.

Front cover member 28 may consist of a perimeter frame 34 which is designed to be fitted over, for frictional mating onto, the corresponding end wall 16. Frame 34 may be additionally secured thereto by welding or the like. Perimeter frame 34 has a rectangular aperture 36 generally matching, in both size and location, aperture 26 located in the corresponding end wall 16. Aperture 36 is covered by a screen 40 which is fastened to frame 34 by welding or the like. Screen 40 has a circular opening 42 formed therethrough.

Rear cover member 30 may consist of a perimeter frame 46 similar to frame 34, again so to be frictionally fitted over the corresponding end wall 16 and secured by welding or the like. Louvers 48 and a screen 50 are located within and mounted to perimeter frame 46. Louvers 48 are inclined upwardly so that ambient ultraviolet rays emitted rearwardly from UV device 20 will be blocked or deflected upwardly away from, surface 8 upon which housing 10 rests, thereby preventing ultraviolet degradation of adjacent areas of surface 8 such as exposed carpets. Lower wall 12b and side walls 14 of housing 10 are ultraviolet impervious.

Housing 20b of UV device 20 is spaced inwardly from the front end wall of end walls 16. Turbine baffles 21 are mounted in the upstream end of housing 20b so as to slow airflow entering the housing. A coupling tube 54 is mounted onto housing 20b so as to extend from housing 20b to opening 42. Housing 20b and tube 54 are co-axial with opening 42. Tube 54 may be mounted to housing 20b by welding or bolting or the like. External tube 56 is inserted through opening 42 in screen 40 to frictionally mount into tube 54.

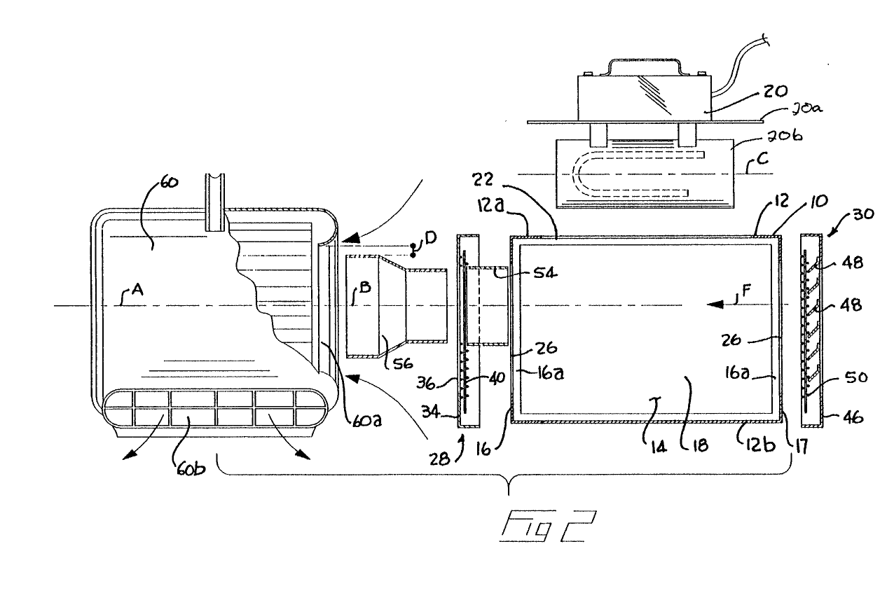

In one embodiment of the present invention housing 10 is positioned for use by placing the free end of tube 56 adjacent the air inlet 60a of a standard commercially available high mass-flow rate air blower 60. Blower fan axis A, axis B of tubes 54 and 56 and the longitudinal axis C of UV device 20 are aligned generally coaxially. In operation blower 60 draws air in direction F through housing 10, into inlet 60a and discharges it from an outlet 60b. Such commercially available blowers are normally provided with an internal ‘squirrel-cage-type’ fan which produces a maximum air intake velocity and therefore maximum intake mass-flow rate around the perimeter of inlet 60a. End 56a of tube 56 is sized so that it does not cover inlet 60a entirely but, rather, leaves the perimeter of inlet 60a exposed. The radial dimension of the exposed area of inlet 60a is designated as dimension D in FIG. 2. In one embodiment dimension D is generally 1 inch (2.5 cm). The air intake velocity through inlet 60a decreases significantly radially closer to blower fan axis A. External tube 56 may be flared outwardly so that free end 56a is shaped like a diffuser. Thus when tube 56 is positioned or mounted so as to place end 56a adjacent to, or to nest end 56a slightly within inlet 60a, end 56a will only cover the area of inlet 60a having the lowest air intake mass-flow rate while leaving unobstructed the exposed annular perimeter of inlet 60a having dimension D which corresponds to the inlet area having high mass-flow rate.

In the applicant's experience, commercial blowers 60 have side inlets 60a of approximately 9 inches (23 cm) in diameter. To cover approximately 60% by area of the area of inlet 60a, and 56a of external tube 56 should therefore be approximately 7 inches (18 cm) in diameter. By this arrangement (or like ratios of dimensions) the air flow velocity past the ultraviolet source within UV device 20 is maintained within parameters which result in a satisfactory reduction of both chemical and biological contaminants on each pass as air is recirculated in the room, mixed within the air mass in the room and past through UV device 20 repeatedly over the decontamination period, for example the period over which the blower would normally be used to dry a room.

In FIG. 6 an alternative ultraviolet radiation emitting device 20′ is illustrated which has a significantly larger diameter than the illustrated UV device 20. In this embodiment external tube 56′ is not flared at end 56a′.

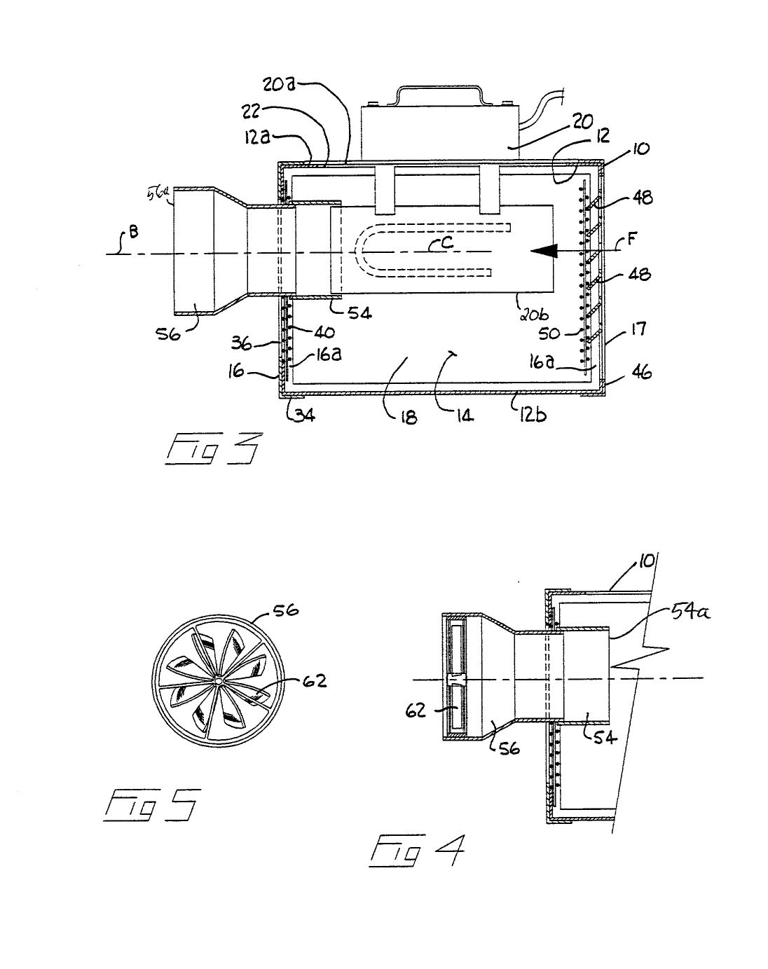

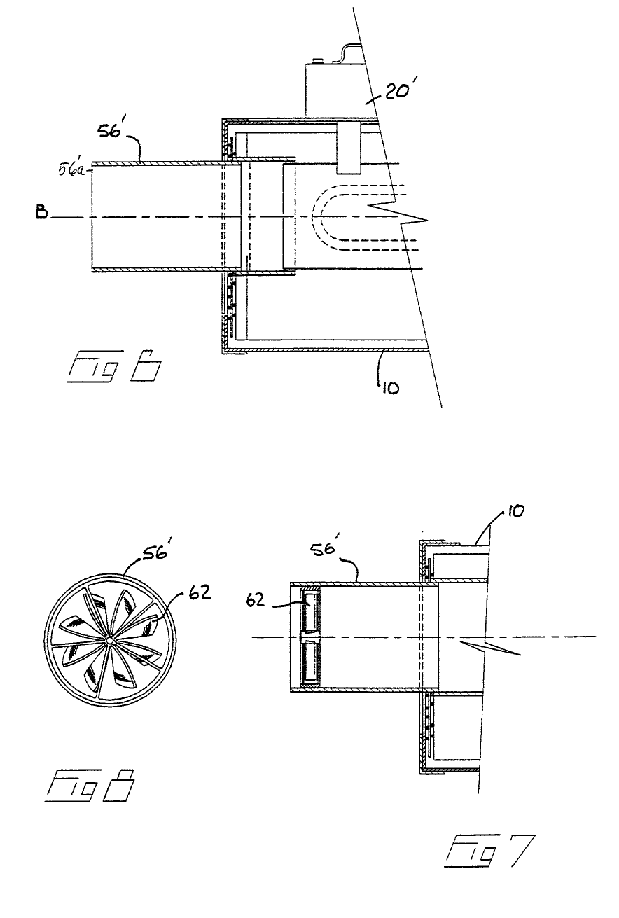

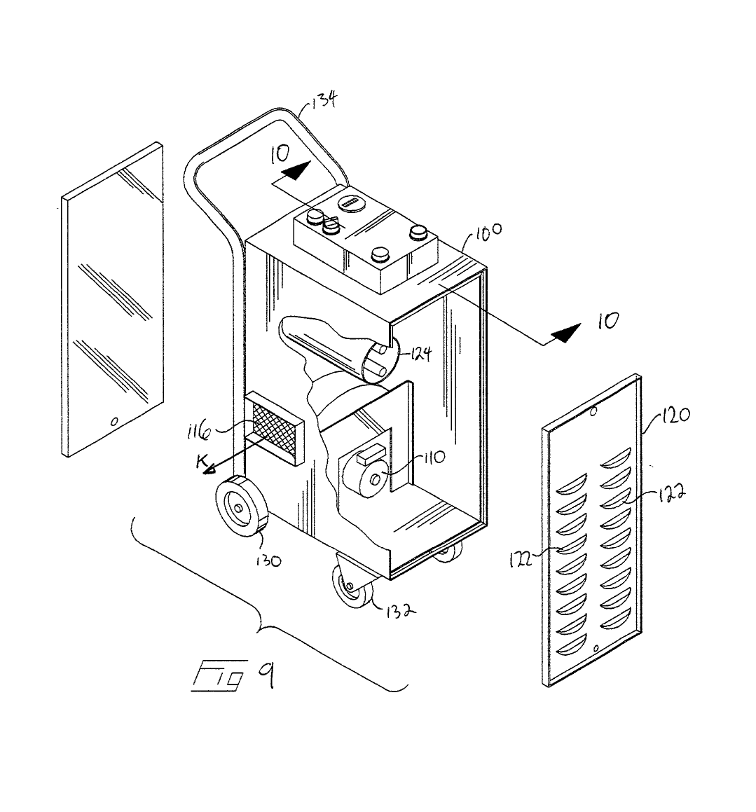

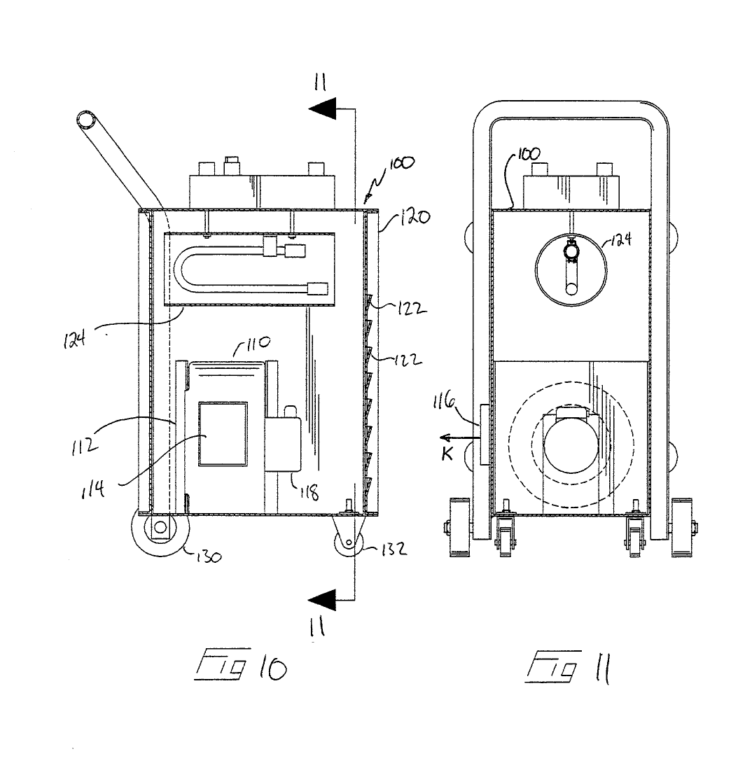

In an alternative embodiment of the invention as illustrated in FIGS. 4, 5, 7 and 8, the air purifier of the present invention may be used without the co-operating use of commercial blower 60. In this embodiment an electrically operated fan or impeller 62 is mounted within a housing containing UV device 20. Although representatively illustrated as mounted within external tube 56 or 56′ so as to draw air to be decontaminated past the ultraviolet source in UV device 20 and to expel the air from the tube, it may in one embodiment be a fan, including the blower-type mounted to the inlet side of housing 10 so long as the appropriate air flow rates may be attained by drawing a low mass-flow rate through UV device 20 and by ducting or by passing around UV device 20 the balance of the mass-flow rate making up the high mass-flow rate of the fan, blower or other air prime-mover. Thus in FIG. 4, end 54a of tube 54 is spaced from UV device 20 so as to draw air from around UV device 20 and air through UV device 20.

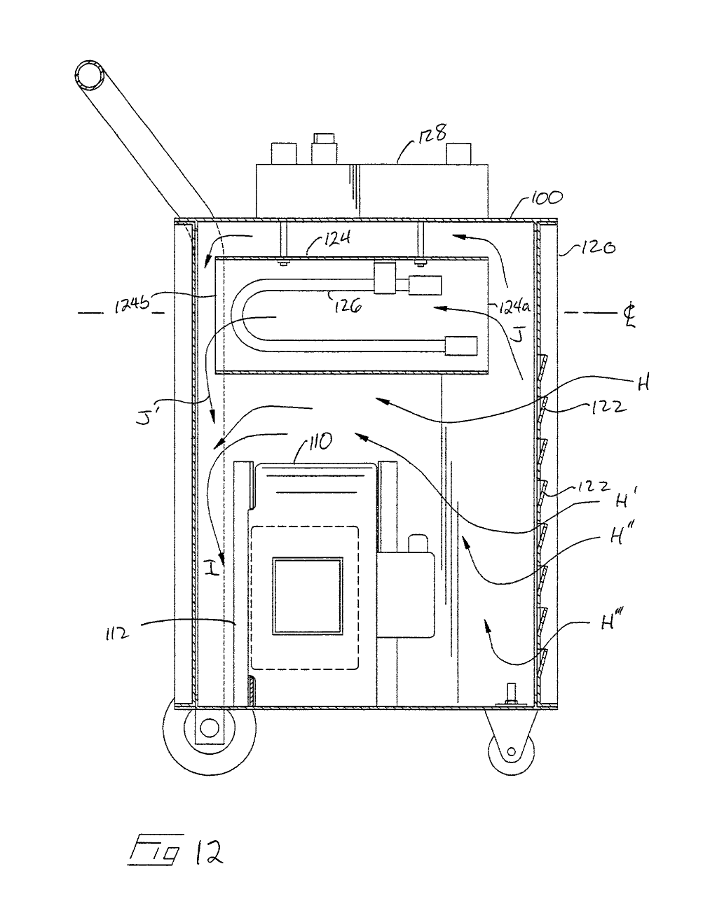

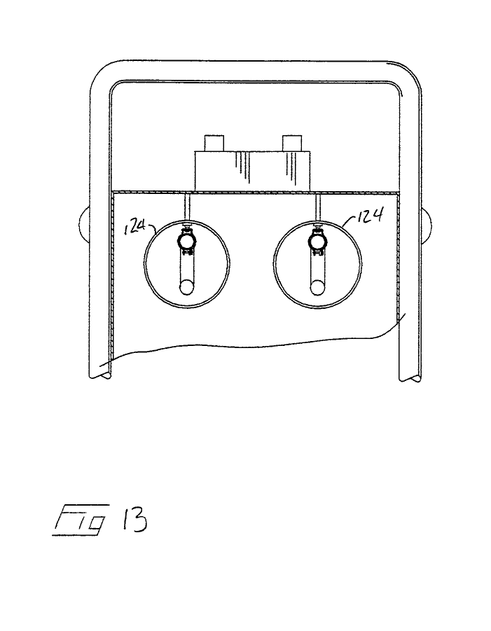

It is also intended to be within the scope of the present invention for housing 10 to be rigidly mounted to blower 60, for example encapsulated within a single rigid housing. Thus as seen in FIGS. 9-12, in a further embodiment of the present invention, housing 100 has a blower 110 mounted on the floor of the housing. The inlet 112 of the blower is enclosed within the housing. The outlet 114 of the blower directs air from the blower out through vent 116 in a sidewall of housing 100. The fan (not shown) within blower 110 is driven by motor 118.

Ventilated panel 120 forms a front wall of housing 100 and is mounted in opposed relation to inlet 112 on blower 110. With the exception of vent 116, the remainder of the walls of housing 100, the floor of the housing and the roof of the housing form a sealed enclosure so that ambient air is drawn in through vents 122 on ventilated panel 120. Thus, for example, ambient air mass to be drawn in directions H, H′, H″ and H″′ as better seen in FIG. 12 into the housing cavity and around blower 110 so as to be drawn into inlet 112 in direction I of the suction generated at the inlet by the blower.

An ultraviolet-emitting device, hereinafter referred to as UV device 124, is mounted to housing 100 so as to be rigidly suspended within the housing spaced apart above blower 110. Blower 110 may be a 500 cfm capacity blower. UV device 124 may be similar to UV device 20 so that ambient air passing into housing 100 through vents 122 may be drawn in direction J into the inlet 124a of UV device 124 in a relatively low mass-flow rate flow past ultraviolet emitters 126 mounted within the air flow path within UV device 124.

The low mass-flow rate air flow in direction J past UV emitters 126 is in sufficiently close proximity to, for a sufficiently long duration or dwell time within the effective range of UV emitters 126 so that mold, bacteria and other impurities are cleansed by the operation of UV emitters 126 prior to the air flow exiting outlet 124b in direction J′.

The purified air flow leaving UV device 124 in direction J′ is drawn towards and into inlet 112 by the suction generated by blower 110, the air flow from UV device 124 co-mingling with the ambient air flow in direction I. The co-mingled air flow enters blower 110 through inlet 112 so as to form the high mass-flow rate air flow exiting in direction K through vent 116.

Electronic controls 128 for the operation of blower 110 and UV device 124 may be mounted atop housing 100 for ease of use. Housing 100 may be mounted on wheels 130 and/or casters 132 and may be provided with a handle 134 so as to facilitate transportation and placement.

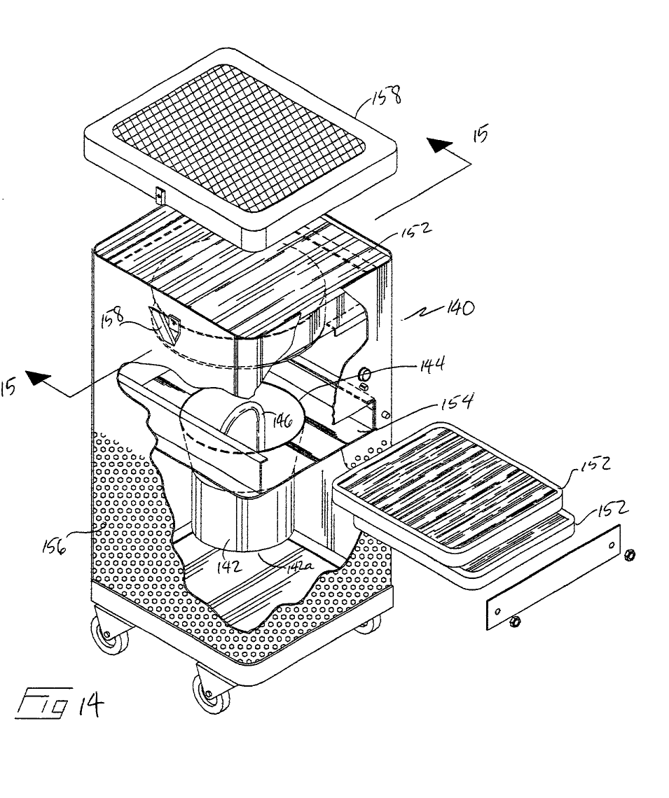

Alternative arrangements are also intended to be within the spirit and scope of the present invention. For example, as seen in FIG. 13, housing 110 may contain a plurality of low mass-flow rate UV devices 124 mounted spaced apart from one another and spaced apart from blower 110. The cumulative effect of a plurality of UV devices 124 allows for the use of a higher mass-flow rate blower 110, for example an approximately 1200 cfm blower if two UV devices 124 are employed, while still maintaining a mass-flow rate ratio of the cumulative mass-flow rate through UV devices 124 relative to the high mass-flow rate through blower 110 so as to purify or decontaminate a sufficiently high percentage of the high mass-flow rate through blower 110. Thus, within a reasonable time frame, that is, within a reasonable number of air turn-overs, all of the air within the enclosure being treated will have passed through UV devices 124. It is also intended to be within the scope of the present invention that the relationship of UV devices 124 to blower 110 are reversed so that the blower is mounted in the upper regions of housing 100 and the UV devices 124 are mounted in the lower regions of the housing.

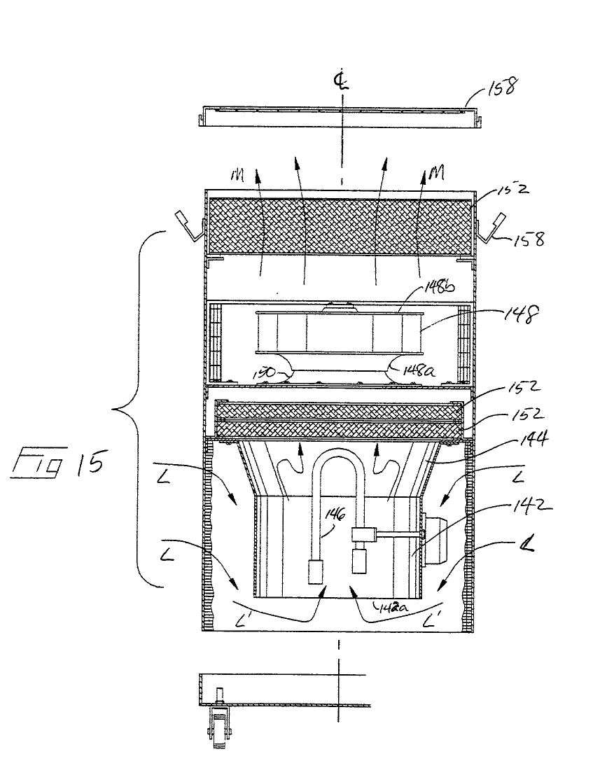

In a further alternative embodiment, as seen in FIGS. 14 and 15, a housing 140 contains a duct 142 rigidly mounted to a downstream diffuser 144, the duct and diffuser containing an ultraviolet emitter, hereinafter UV device 146, rigidly mounted cantilevered within the duct and diffuser.

Duct 142 and diffuser 144 are mounted co-linearly with a downstream blower or fan 148. Fan 148 has an inlet 148a communicating with a converging inlet duct 150 mounted in opposed facing relation to diffuser 144 on opposite sides of, so as to sandwich between, removable particulate filters 152.

Particulate filters 152 may be mounted between diffuser 144 and converging duct 150 on a rack or shelf 154 perpendicularly across the air flow path of air being drawn through duct 142, diffuser 144, duct 150 and into fan 148. Fan 148 creates a suction at its inlet 148a so as to draw ambient air through ventilated side walls 156 in direction L and into duct 142 through inlet 142a in direction L′. The air is then drawn past UV device 146, slowing through diffuser 144 thereby increasing the dwell time and exposure of the mass-flow to UV device 146 before the air flow passes through filters 152 into the inlet of fan 148. Fan 148 then forces the air flow from outlet 148b in direction M through a further filter 152 so as to exit from housing 140 through cover 158, cover 158 being retained on side walls 156 by latches 158. Again, it is intended to be within the scope of the invention that a plurality of UV devices 146 are mounted within duct 142 and diffuser 144.

What is claimed is:

1. An air purifier comprising:
a high mass-flow rate air-mover having an inlet and an outlet,
a low mass-flow rate ultra-violet radiation decontamination device having an inlet and an outlet,
said decontamination device mounted in spaced relation to said air-mover so as to space said inlet of said air-mover from said inlet of said decontamination device,
said inlet of said decontamination device disposed oppositely to said inlet of said air-mover relative to said outlet of said decontamination device along a low mass-flow rate flow path of a low mass-flow rate air flow passing ambient air into said inlet of said decontamination device, through said decontamination device so as to pass for an operative dwell time into operative proximity to at least one UV emitter mounted in said decontamination device and so as to exit from said outlet of said air-mover to then be drawn into said inlet of said air-mover,
said air-mover spaced from said decontamination device so as to draw into said inlet of said air-mover a second air-flow of said ambient air, said second air flow flowing along a second flow path wherein said second flow path does not flow within said operative proximity to said at least one UV emitter.
wherein said low mass-flow rate air flow and said second air flow cumulatively form a high mass-flow rate flow being urged by said air-mover through said air-mover so as to expel said high mass-flow rate air flow into said ambient air.

2. The air purifier of claim 1 wherein said air-mover and said decontamination device are rigidly mounted vertically spaced from one another within a housing.

3. The air purifier of claim 2 wherein said decontamination device is mounted above said air-mover.

4. The air purifier of claim 2 wherein said decontamination device is mounted below said air-mover.

5. The air purifier of claim 3 wherein said air-mover is a blower and said UV emitter is mounted within a duct.

6. The air purifier of claim 4 wherein said air-mover is a blower and said UV emitter is mounted within a duct.

7. The air purifier of claim 2 wherein said housing has an outlet aperture in a first surface of said housing and at least one inlet aperture in a second surface of said housing, said housing otherwise being substantially sealed to air flow, said outlet aperture communicating with said outlet of said air-mover, said at least one inlet aperture adjacent said inlet of said decontamination device, said at least one inlet aperture communicating ambient air flow of said ambient air into both said second flow path and said low mass-flow rate flow path.

8. The air purifier of claim 7 wherein said outlet aperture does not face in the same direction as said at least one inlet aperture whereby air flow from said outlet aperture exhausting into said ambient air may recirculate within the air space of an enclosure in which said air purifier is placed before being re-drawn as said ambient air flow into said at least one inlet aperture.

9. The air purifier of claim 8 wherein said air-mover is a blower and said UV emitter is mounted within a duct.

10. The air purifier of claim 9 wherein said duct is aligned so that said low mass-flow rate air flow through said duct is orthogonal to a plane substantially containing said inlet of said air-mover.

11. The air purifier of claim 10 wherein said duct is elongate and aligned orthogonally to an exhaust direction of said blower.

12. The air purifier of claim 11 wherein said at least one inlet aperture is generally perpendicular to said outlet aperture.

13. The air purifier of claim 12 wherein said at least one inlet aperture and said exhaust aperture are on side walls of said housing.

14. The air purifier of claim 13 wherein said at least one inlet aperture is an array of apertures vertically spaced over one sidewall of said sidewalls of said housing.

15. The air purifier of claim 7 wherein said second air flow and said low mass-flow rate air flow are generally parallel at positions along said air flows when said low mass-flow rate air flow is in said operative proximity to said at least one UV emitter.

16. The air purifier of claim 7 wherein said second air flow and said low mass-flow rate air flow are ducted in a common duct containing said at least one UV emitter.

17. The air purifier of claim 15 wherein said decontamination device and said air-mover are co-axial along the substantial air flow directions of said second air flow, said low mass-flow rate air flow, and an exhaust air flow direction from said outlets of said air-mover and said decontamination device.

18. The air purifier of claim 16 wherein said decontamination device and said air-mover are co-axial along the substantial air flow directions of said second air flow, said low mass-flow rate air flow, and an exhaust air flow direction from said outlets of said air-mover and said decontamination device.

19. The air purifier of claim 17 wherein said air-mover is mounted above said decontamination device.

20. The air purifier of claim 18 wherein said air-mover is mounted above said decontamination device.
